(19) United States
(12) Patent Application Publication
(10) Pub. No.: US 2022/0313597 A1
(43) Pub. Date: Oct. 6, 2022

(54) THERMOGELLING CANNABINOID COMPOSITION AND METHOD OF MANUFACTURE AND USE THEREOF

(71) Applicant: HEXO OPERATIONS INC., Ottawa (CA)

(72) Inventors: Jamie Savard, Gatineau (CA); Francois Chouinard, Blainville (CA); George Elvira, Montreal (CA)

(21) Appl. No.: 17/616,992

(22) PCT Filed: Jun. 5, 2020
(86) PCT No.: PCT/CA2020/050780

§ 371 Date: Dec. 6, 2021

Related U.S. Application Data
(60) Provisional application No. 62/858,112, filed on Jun. 6, 2019.

(51) Int. Cl. A61K 9/00; A61K 31/05; A61K 31/352; A61K 47/34; A61K 47/06; A61K 9/107; A61K 47/44; A61K 47/14

(52) U.S. Cl. CPC A61K 9/006; A61K 31/05; A61K 31/352; A61K 47/34; A61K 47/06; A61K 9/107; A61K 47/44; A61K 47/14

(57) ABSTRACT

The present disclosure relates to a cannabinoid composition that has enhanced absorption rates or higher concentrations of the administered cannabinoid into the systemic circulation, the composition comprising at least one cannabinoid and a viscosity modifier present in an effective amount to change the composition from a liquid at about room temperature to a gel upon increase to about body temperature. The disclosure also relates to methods of use of the compositions and cannabinoid products comprising such compositions.

### CROSS-REFERENCE TO RELATED APPLICATION

The present application claims the benefit of U.S. provisional patent application Ser. No. 62/858,112 filed on Jun. 6, 2019. The contents of the above-referenced document are incorporated herein by reference in their entirety.

### TECHNICAL FIELD

The present disclosure relates to thermogelling cannabinoid compositions that are suitable for transmucosal administration of the cannabinoid. Method of making and using such compositions are also encompassed by the present disclosure.

### BACKGROUND

Cannabis produces desirable physiological effects associated with a feeling of physical and/or emotional satisfaction and/or can be useful in the treatment of variety of diseases and conditions (e.g., pain, anxiety, inflammatory disorders, immune disorders, metabolic disorders, and the like). Two commonly used cannabinoids for recreational use and/or in the health/wellness field are Cannabidiol (CBD) and Tetrahydrocannabinol (THC).

Administration of cannabis has evolved over time from inhalation of combustible by-products (i.e., smoking) to other preferred routes of administration, such as spray administration. Cannabis formulations intended for use in spray administration devices are typically formulated with a carrier oil (e.g., triglyceride oil such as “medium chain triglyceride” (MCT)), as opposed to aqueous formulations, due to cannabinoids being highly lipophilic and having poor aqueous solubility. The purpose of the carrier oil is to aid in solubilizing the hydrophobic cannabinoid in the formulation. However, there are at least two main disadvantages with such oily formulations.

Firstly, absorption of oily formulations via transmucosal delivery is suboptimal, especially when compared with aqueous formulations; this reduces the efficacy of such administration. For example, with oral administration, the absorption may be substantially reduced because the oil is not soluble in water-based saliva. Alternatively, with intranasal administration, absorption may be similarly reduced due to the sprayed formulation passing through the nasal cavity and being swallowed.

Secondly, oily formulations can often give rise to leakage problems in spray administration devices due to the relatively high viscosity of the liquid. It has been observed that oily formulations tend to “string” once spraying has stopped. “Stringing” is the phenomenon wherein the liquid composition remains attached to the opening of the spray devices and forms a “capillary” between the opening and the exterior environment. As a result of the stringing, the oily formulation may leak under the influence of gravity onto the storage surfaces. Alternatively, the oily formulation left around and inside the opening of spraying devices tends to dry and form a crust. If the crust is allowed to build-up, then it eventually blocks the opening.

Previous attempts to address the aforementioned absorption problem involved increasing the residence time of the oil formulations by increasing its viscosity. However, this solution has an aggravating effect on the rheology profile of the composition contributing to the leakage problem, which leads to an inherent trade-off in known cannabis formulations that inadequately address both issues.

Thus, there remains a need for a cannabinoid composition having improved transmucosal administration of the cannabinoids, yet still minimizing the leakage problems as discussed above.

### SUMMARY

This Summary is provided to introduce a selection of concepts in a simplified form that are further described below in the Detailed Description. This Summary is not intended to identify key aspects or essential aspects of the claimed subject matter.

The inventors have developed cannabinoid compositions that are surprisingly capable of overcoming the disadvantages as described above. In particular, the present disclosure is directed to cannabinoid compositions that are liquid at about room temperature and turn into a gel when increased to about body temperature. The compositions of the present disclosure allow for enhanced residence time of intimate contact with the mucosal surfaces after application and provide increased water solubility. Due to their lower viscosity, the compositions of the present disclosure also minimize the leakage problems whilst the compositions are in the container before its application.

As embodied and broadly described herein, the present disclosure relates to a composition comprising (i) an emulsion including a cannabinoid component, the cannabinoid component including at least one cannabinoid and (ii) a viscosity modifier, the composition being in a liquid state at about room temperature, the viscosity modifier operating to increase the viscosity of the composition such that the composition is in a semi-solid or solid state at about body temperature.

As embodied and broadly described herein, the present disclosure also relates to a composition comprising (i) an emulsion including a cannabinoid component, the cannabinoid component including at least one cannabinoid and (ii) a viscosity modifier, the composition being in a free-flowing state at about room temperature, the viscosity modifier operating to increase the viscosity of the composition at about body temperature such that the composition forms a hydrogel.

As embodied and broadly described herein, the present disclosure also relates to a composition comprising (i) an emulsion including a cannabinoid component, the cannabinoid component including at least one cannabinoid and (ii) a mucoadhesive agent, the mucoadhesive agent operating to increase the viscosity of the composition upon contacting a surface of a mucosa such that the composition forms a hydrogel having mucoadhesive properties.

As embodied and broadly described herein, the present disclosure also relates to a thermogelling cannabinoid composition comprising: a) at least one cannabinoid; b) a viscosity modifier present in an amount effective to change the composition from a liquid at about room temperature to a gel upon increase to about body temperature; and c) a pharmaceutically acceptable excipient comprising water.

As embodied and broadly described herein, the present disclosure also relates to a cannabinoid product for transmucosal administration, preferably to an oral or nasal mucosa, of a subject, wherein the product comprises a composition as described herein. The product may be provided as a sublingual liquid spray, a buccal liquid spray, or a nasal liquid spray.

As embodied and broadly described herein, the present disclosure also relates to a method of treating a disease or condition in a subject comprising applying to a mucosa, preferably an oral and/or nasal mucosa, of the subject a composition as described herein.

In some embodiments, the at least one cannabinoid may be present in an amount of from about 0.001 mg/mL to about 100 mg/mL. The at least one cannabinoid may be provided as an isolated cannabinoid having >75%, preferably >80%, preferably >90%, preferably >95%, preferably >98%, preferably >99%, or preferably >99.5% purity; the isolated cannabinoid may be present in at least one carrier oil. The at least one cannabinoid may be cannabidiol (CBD), tetrahydrocannabinol (THC), or a mixture thereof; when the at least one cannabinoid is a mixture of THC and CBD, the (w/w) ratio of THC:CBD may be between about 1:1000 and about 1000:1. The at least one cannabinoid is provided as a cannabinoid component, which may comprise the at least one cannabinoid, or a mixture of the at least one cannabinoid and a carrier oil. The cannabinoid component may be present in an amount of from about 0.1% to about 10% by weight of the composition.

In some embodiments, the composition may comprise the cannabinoid component and the viscosity modifier in a ratio (w/w) of cannabinoid component:viscosity modifier within 0.9-1.1:1-9.5. For example, when the composition includes about 1 wt. % of the cannabinoid component, the ratio (w/w) of cannabinoid component:viscosity modifier can be within 0.9-1.1:7.9-9.5. Alternatively, when the composition includes about 3 wt. % of the cannabinoid component, the ratio (w/w) of cannabinoid component:viscosity modifier can be within 0.9-1.1:3.2-3.5. Alternatively, when the composition includes about 5 wt. % of the cannabinoid component, the ratio (w/w) of cannabinoid component:viscosity modifier is within 0.9-1.1:1-1.2.

In some embodiments, the viscosity modifier may be a poly(propylene oxide)/poly(ethylene oxide) copolymer. The copolymer may have a molecular weight of from about 7,000 g/mol to about 18,000 g/mol, and an ethylene oxide content of from about 30% to about 90% by weight of the copolymer. The viscosity modifier may comprise a block co-polymer selected from the group consisting of: Poloxamer 108, Poloxamer 124, Poloxamer 182, Poloxamer 183, Poloxamer 184, Poloxamer 185, Poloxamer 188, Poloxamer 212, Poloxamer 217, Poloxamer 237, Poloxamer 217, Poloxamer 288, Poloxamer 331, Poloxamer 335, Poloxamer 338, Poloxamer 407 and a mixture thereof. In a specific embodiment, the viscosity modifier is Poloxamer 407, or a mixture of Poloxamer 407 and Poloxamer 188; such a mixture of Poloxamer 407 and Poloxamer 188 may comprise at least 85% Poloxamer 407. The viscosity modifier may be present in an amount of from about 0.1% to about 15% by weight of the composition.

In some embodiments, the composition as described above may further comprise at least one surfactant. In some embodiments, the composition may comprise the cannabinoid component and the at least one surfactant in a ratio (w/w) of cannabinoid component:surfactant within 0.9-1.1:1.9-2.2. For example, when the composition comprises about 1 wt. % of the cannabinoid component, the ratio (w/w) of cannabinoid component:surfactant is within 0.9-1.1:1.4-1.9. Alternatively, when the composition comprises about 3 wt. % of the cannabinoid component, the ratio (w/w) of cannabinoid component:surfactant can be within 0.9-1.1:1.8-2.2. Alternatively, when the composition comprises about 5 wt % of the cannabinoid component, the ratio (w/w) of cannabinoid component:surfactant can be within 0.9-1.1:1.9-2.2.

In some embodiments, the at least one surfactant may be a polyethylene glycol derivative of castor oil; in a specific embodiment, the at least one surfactant may be PEG-40 hydrogenated castor oil. The composition may comprise the at least one surfactant in an amount of from about 0.01 wt. % to about 15 wt %.

In some embodiments, the composition as described above may further comprise at least one permeation enhancer. The composition may comprise about the at least one permeation enhancer in an amount of about 0.5 wt. % to about 15 wt. %.

In some embodiments, the composition as described above may further comprise at least one mucoadhesive agent. The at least one mucoadhesive agent may be selected from the group consisting of xanthan gum, gellan gum, arabic, guar gum, tragacanth gum, locust bean gum, methylcellulose, carboxymethyl cellulose, hydroxyethyl cellulose, hypromellose, gelatin, carrageenan, agar, pectin, polycarbophil, Noveon AA-1, Carbopol compounds, carbomer compounds, and a combination thereof.

In some embodiments, the composition as described above may further comprise at least one flavorant. The at least one flavorant may be selected from the group consisting of extracts of cinnamon, cucumber, mint, orange, lime, citrus, cookie dough, chocolate, vanilla, jasmine, lychee, almond, banana, grape, pear, pineapple, pine, oak, apple, pumpkin, grapefruit, watermelon, cotton sugar, durian, longan, taro, sapote, toffee nut, caramel, lotus, mango, mangosteen, coconut, coffee, strawberry, passion fruit, blueberry, raspberry, kiwi, walnut, cocoa, cherimoya, custard apple, papaya, fig, plum, nectarine, peaches, guava, honeydew, jackfruit, kumquat, loquat, palm, pomelo, persimmon, quince, tamarind, and any combinations thereof. In another embodiment, the flavorant may comprise a terpene.

In some embodiments, the composition as described above may be an oil-in-water emulsion comprising the cannabinoid; in one embodiment, the composition may be a self-emulsifying oil-in-water emulsion. The emulsion formed by the composition may comprises oil droplets having an average mean particle size of <200 nm, preferably <150 nm, preferably <100 nm, preferably <75 nm, preferably <70 nm, preferably <60 nm, or preferably <50 nm. In one example, the emulsion comprises oil droplets having a D90 of <200 nm, preferably <150 nm, preferably <100 nm, preferably <75 nm, preferably <70 nm, preferably <60 nm, or preferably <50 nm; alternatively, the emulsion comprises oil droplets having a D50 of <200 nm, preferably <150 nm, preferably <100 nm, preferably <75 nm, preferably <70 nm, preferably <60 nm, or preferably <50 nm.

In some embodiments, the composition described herein may have an initial viscosity of less than about 750 mPas at about room temperature, and an applied viscosity of more than about 5,000 mPas at about body temperature. In another example, the composition described herein may have an initial viscosity of less than about 200 mPas at about room temperature, and an applied viscosity of more than about 10,000 mPas at about body temperature.

In some embodiments, the composition described herein may exhibit a residence time on the surface of the mucosa of greater than about 10 seconds, preferably greater than about 20 seconds, preferably greater than about 30 seconds, preferably greater than about 40 seconds, preferably greater than about 50 seconds, preferably greater than about 1 minute, preferably greater than about 2 minutes, preferably greater than about 5 minutes, preferably greater than about 10 minutes, preferably greater than about 20 minutes, following application on the mucosal surface.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1 is a graph showing the gelation curve of a composition in accordance with an embodiment of the present disclosure obtained at a shear rate of 1 s⁻¹. The crossover point between the storage modulus (▪) and the loss modulus (▴) indicates a phase change from liquid to gel form (at 34° C.);

FIG. 2 is a graph showing the viscosity of a composition in accordance with an embodiment of the present disclosure at 25° C. (▪) and at 40° C. (●). The viscosity of the solution is shear independent at 25° C. but shear dependent upon gelation at 40° C.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Compositions

In one broad aspect, the present disclosure relates to a cannabinoid composition that has enhanced bioavailability of the administered cannabinoid upon its application to the mucosa. As used herein, the term “composition” includes an aqueous composition. The composition may be in the form of a liquid. The term “mucosa” refers to the mucosal cells that line the various cavities in the body wherein the composition is administered for absorption into the systemic circulation. Suitable examples include, but are not limited to, oral mucosa, nasal mucosa, vaginal mucosa, ocular mucosa, and rectal mucosa. Preferably, the compositions of the present invention are for application to the oral mucosa, nasal mucosa, or both. “Oral mucosa” means mucosal cells lining the oral cavity, and includes the mucous membrane beneath the tongue (i.e., sublingual) and/or the buccal mucosa at the inside of the cheek and gum. “Nasal mucosa” means mucosal cells lining the nasal cavity. “Vaginal mucosa” means the vaginal mucous membrane. “Ocular mucosa” means mucosal cells lining the ocular cavity. “Rectal mucosa” means the inner mucosal cells lining the rectum.

As previously stated, the compositions of the present disclosure lead to enhanced bioavailability of the cannabinoid when applied to the mucosa. This is achieved by formulating the cannabinoid with a viscosity modifier present in an amount effective to change the composition from a liquid at about room temperature to a gel upon increase to about body temperature. Essentially, the transition of the composition from liquid to gel form happens upon contact with the mucosa due to the temperature change. As such, the composition in the liquid form has relatively low viscosity prior to and during its application, thereby mitigating against leakage problems associated with dispensing prior art compositions. The resultant gel form of the presently disclosed composition has relatively higher viscosity than its liquid form. As a result of its increased viscosity, the gel form increases the residence time of the composition on the surface of the mucosa. Oral absorption is faster than enteric absorption, and increased residence time on the surface of the mucosa allows for a greater amount of cannabinoid to be absorbed via this route, thereby increasing the bioavailability of the administered cannabinoid. The net outcome is enhanced transmucosal delivery of the cannabinoid into the systemic circulation (i.e., blood stream) due to a longer residence time of the gel composition on the mucosal surface.

Accordingly, one aim of the present disclosure is to provide a composition that has enhanced residence time of said composition on the mucosa following its application. Another aim of the present disclosure is to provide a composition that has enhanced absorption rates of the administered cannabinoid into the systemic system. A further aim of the present disclosure is to provide a composition having higher concentrations of the administered cannabinoid absorbed into the systemic system. A yet further aim of the present disclosure is to provide a composition suitable for administration of a therapeutically effect amount of the administered cannabinoid to treat a disease or condition in a subject. A yet further aim of the present disclosure is to provide a composition suitable for administration of an effective amount of the administered cannabinoid to produce a feeling of physical and/or emotional satisfaction in a subject. It is desirous that the composition has a rheology profile which helps to substantially reduce or prevent leakage in the administration device. It is also desirous that the composition has stable quality of end product (e.g., bioavailability, shelf-life and/or phase stability).

In the context of the present disclosure, the compositions are characterized as being “thermogelling”, which broadly has the meaning that the compositions can change form based on a temperature that the compositions are exposed to, e.g., from liquid to a semi-solid hydrogel or solid gel. For example, the compositions of the present disclosure are generally liquid with lower viscosity at about room temperature, which can be from about 20° C. to 25° C., but changes to a gel with higher viscosity when exposed to body temperature, which can be about 37° C. to 40° C. It should be noted that the transition between liquid and gel does not necessarily need to be at body temperature, but preferably the compositions shall undergo transition in the interval between about 30° C. to about 37° C. In a preferred embodiment, the transition is sufficiently distinct at a defined temperature or at a fairly narrow temperature interval.

The compositions of the present disclosure may form a cohesive viscous gel at about body temperature so that the composition remains on the mucosal surfaces for a longer duration after application. The preferred cohesive viscous gel is also able to better retain its shape and resist deformation to enhance delivery/bioavailability of the administered cannabinoid.

In one aspect, the composition of the present disclosure upon application on the surface of a mucosa is converted from a liquid to a gel thereby providing intimate contact with the absorption site for an increased residence time, thus enhancing bioavailability of the applied cannabinoid in the composition. Preferably the bioavailability of the applied cannabinoid in the composition in the gel form is at least about 0.5, at least about 1.5 or at least about 2 times greater than the bioavailability of the applied cannabinoid of the composition in liquid form. As used herein, the term “bioavailability” means the physiological availability of a given amount of the cannabinoid as distinct from its chemical potency proportional to the administered amount that is absorbed into the systemic circulation (i.e., bloodstream).

### Cannabinoid

The composition of the present disclosure comprises at least one cannabinoid. As used herein, the term “cannabinoid” is generally understood to include any chemical compound that acts upon a cannabinoid receptor. Cannabinoids are commonly used for recreational purposes to produce physiological effects associated with a feeling of physical and/or emotional satisfaction. Cannabinoids can also be useful in the treatment and/or prophylaxis of a wide variety of diseases or conditions, such as pain, anxiety, inflammation, autoimmune diseases, neurological disorder, psychiatric disorder, malignancy, metabolic disorder, nutritional deficiency, infectious disease, gastrointestinal disorder, or cardiovascular disorder. Cannabinoids may also have application as neuroprotectants, for example, in limiting neurological damage following ischemic insults, such as stroke and trauma, or in the treatment of neurodegenerative diseases such as Alzheimer's disease, Parkinson's disease and HIV dementia. Cannabinoids for inclusion in the compositions of the present disclosure include phytocannabinoids (i.e., found in cannabis and some other plants) and synthetic cannabinoids (i.e., manufactured artificially).

Examples of suitable phytocannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ⁷-cis-iso tetrahydrocannabivarin, Tetrahydrocannabinol (THC), Δ⁹-tetrahydrocannabinol-C4, Δ⁹-tetrahydrocannabinolic acid-C4 (THCA-C4), Δ⁹-tetrahydrocannabionolic acid B (THCA-B), Δ⁹-tetrahydrocannabiorcol (THC-C1), Δ⁹-tetrahydrocannabivarin (THCV), tetrahydrocannabivarinic acid (THCVA), ethoxy-cannabitriolvarin (CBTVE), trihydroxy-Δ⁹-tetrahydrocannabinol (triOH-THC), 10-ethoxy-9hydroxy-Δ^{6a}-tetrahydrocannabinol, 8,9-dihydroxy-Δ^{6a}-tetrahydrocannabinol, 10-oxo-Δ^{6a}-tetrahydrocannabionol (OTHC), 3,4,5,6-tetrahydro-7-hydroxy-α-α-2-trimethyl-9-n-propyl-2,6-methano-2H-1-benzoxocin-5-methanol (OH-iso-HHCV), Δ^{6a,10a}-tetrahydrocannabinol (Δ^{6a,10a}-THC), Δ⁸-tetrahydrocannabivarin (Δ⁸-THCV), Δ⁹-tetrahydrocannabiphorol (Δ⁹-THCP), Δ⁹-tetrahydrocannabutol (Δ⁹-THCB), derivatives of any thereof, and combinations thereof. Further examples of suitable cannabinoids are discussed in at least PCT Patent Application Pub. No. WO2017/190249 and U.S. Patent Application Pub. No. US2014/0271940, which are incorporated by reference in their entirety.

Examples of suitable synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, quinolinyl esters, and combinations thereof.

The cannabinoid in the compositions of the present disclosure may be in an acid form or a non-acid form, the latter also being referred to as the decarboxylated form since the non-acid form can be generated by decarboxylating the acid form. Preferably, where reference is made to a specific cannabinoid, it will be understood that the cannabinoid is in the decarboxylated form.

The cannabinoid in the compositions of the present disclosure may be a single cannabinoid or may be a combination of two or more cannabinoids. In a non-limiting example, the cannabinoid in the compositions of the present disclosure is cannabidiol (CBD), tetrahydrocannabinol (THC), or a mixture thereof.

Cannabidiol (CBD) means one or more of the following compounds: Δ²-cannabidiol, Δ⁵-cannabidiol (2-(6-isopropenyl-3-methyl-5-cyclohexen-1-yl)-5-pentyl-1,3-benzenediol); Δ⁴-cannabidiol (2-(6-isopropenyl-3-methyl-4-cyclohexen-1-yl)-5-pentyl-1,3-benzenediol); Δ³-cannabidiol (2-(6-isopropenyl-3-methyl-3-cyclohexen-1-yl)-5-pentyl-1,3-benzenediol); Δ^{3,7}-cannabidiol (2-(6-isopropenyl-3-methylenecyclohex-1-yl)-5-pentyl-1,3-benzenediol); Δ²-cannabidiol (2-(6-isopropenyl-3-methyl-2-cyclohexen-1-yl)-5-pentyl-1,3-benzenediol); Δ¹-cannabidiol (2-(6-isopropenyl-3-methyl-1-cyclohexen-1-yl)-5-pentyl-1,3-benzenediol); and (7) Δ⁶-cannabidiol (2-(6-isopropenyl-3-methyl-6-cyclohexen-1-yl)-5-pentyl-1,3-benzenediol). In a preferred embodiment, and unless otherwise stated, CBD means Δ²-cannabidiol.

Tetrahydrocannabinol (THC) means one or more of the following compounds: Δ⁸-tetrahydrocannabinol (A8-THC), Δ⁹-cis-tetrahydrocannabinol (cis-THC), Δ⁹-tetrahydrocannabinol (Δ⁹-THC), Δ⁹-tetrahydrocannabinolic acid A (THCA-A). In a preferred embodiment, and unless otherwise stated, THC means one or more of the following compounds: Δ⁹-tetrahydrocannabinol and Δ⁸-tetrahydrocannabinol.

As is known in the art, various cannabinoids can be used in combination to achieve a desired effect in a user. Suitable mixtures of cannabinoids that can be used in the present disclosure include but are not limited to a mixture of tetrahydrocannabinol (THC), and cannabidiol (CBD). Certain specific ratios of cannabinoids may be useful to produce the feeling of physical and/or emotional satisfaction and/or may be useful in the treatment or management of specific diseases or conditions.

In some embodiments, the (w/w) ratio of the THC to the CBD is between about 1:1000 and about 1000:1. Preferably, the (w/w) ratio of THC to CBD in the composition may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

As noted above, various cannabinoids and combinations thereof can be incorporated into the compositions of the present disclosure in varying amounts sufficient to achieve a desired effect in a user, such as a psychoactive effect, a physiological effect, or a treatment of a condition. The compositions of the present disclosure may achieve a psychoactive effect, a physiological effect, or a combination thereof, in a user. By “psychoactive effect”, it is meant a substantial effect on mood, perception, consciousness, cognition, or behaviour of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of the cannabinoid across the mucosa at sufficient amounts to mediate the therapeutic effects.

The compositions of the present disclosure may comprise the at least one cannabinoid in a concentration of from about 0.001 mg/mL to about 100 mg/mL, including any amount therebetween or any ranges therein; in a non-limiting example, the compositions may comprise from about 0.002 mg/mL to about 100 mg/mL, from about 0.1 mg/mL to about 75 mg/mL, or from about 0.1 mg/mL to about 50 mg/mL, including any amount therebetween or any ranges therein, of the at least one cannabinoid. Cannabinoids provided at such an amount in the compositions of the present disclosure can be particularly effective in penetrating the mucosa into the systemic circulation, preferably without having adverse side-effects.

Cannabinoids for use in the present compositions may be obtained from any suitable source material including, but not limited to, cannabis or hemp plant material (e.g., flowers, seeds, trichomes, and kief). The cannabis or hemp plant material may be provided in milled form or may be in the form of cannabis extracts obtained from cannabis or hemp plant material (e.g., resins, waxes and concentrates). As used herein, a “cannabis extract” refers to an extract obtained from a cannabis plant material according to any procedure known in the art; such extracts yield cannabinoids in substantially pure or isolated form. For example, a cannabis extract may be obtained by a process including an extraction step from plant materials using for example heat decarboxylation to convert cannabinoids in their acid forms to neutral forms followed by or after CO₂ extraction (under sub-critical or super-critical conditions), providing a crude extract. The crude extract may then be “winterized”, that is, extracted with ethanol to remove lipids and waxes, as described for example in U.S. Pat. No. 7,700,368, US 2004/0049059, and US 2008/0167483, which are herein incorporated by reference. Optionally, the method for obtaining the cannabis extract may further include purification steps such as a distillation step to further purify, isolate or crystallize one or more cannabinoids, which is referred to herein as a “distillate”; US20160346339, which is incorporated herein by reference, describes a process for extracting cannabinoids from cannabis plant material using solvent extraction followed by filtration, and evaporation of the solvent in a distiller to obtain a distillate. The distillate may be further cut with one or more terpenes. The distillate may be further purified, for example using chromatographic and other separation methods known in the art, to obtain an “isolate”.

In some embodiments, pure or isolated cannabinoids, such as those provided in a cannabis extract, may be combined with water, lipids, hydrocarbons (e.g., butane), ethanol, acetone, isopropanol, or mixtures thereof.

Cannabinoid used in the compositions of the present invention may be an isolated cannabinoid, such as a cannabis extract, having >75% purity (as in the case of a crude extract), or >80% purity (as in the case of a distillate), or >95% purity, as in the case of an isolate). For example, and without wishing to be limiting, the cannabinoid may have >75%, preferably >80%, preferably >90%, preferably >95%, preferably >98%, preferably >98%, preferably >99% or preferably >99.5%, purity. It is especially preferred that the cannabinoids have high purity (i.e., Pharmacopoeia Grade substances, which may be obtained from a natural source or via synthetic means) to enable sufficient solubility in the composition. Solubility is important so that the cannabinoids remain in solution and do not precipitate out over time.

The at least one cannabinoid in the composition of the present disclosure is provided as a cannabinoid component, which may comprise the at least one cannabinoid, or a mixture of the at least one cannabinoid and a carrier oil. The carrier oil may be a triglyceride oil such as a “medium chain triglyceride” (MCT) oil, or any other suitable carrier oil or solvent. Non-limiting examples of carrier oils or solvent suitable for cannabinoids include: borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g., caprylic/capric glycerides), ethanol, acetone, isopropanol, hydrocarbons, and any combination thereof. The at least one cannabinoid is provided in the cannabinoid component in an amount sufficient to achieve the desired final concentration of cannabinoid in the composition.

### Cannabinoid Emulsions

In embodiments of the present disclosure, the compositions comprising the cannabinoids may be formulated as an emulsion. As is known in the art, an emulsion is a mixture of two liquids, where one is present as microscopic droplets (dispersed phase) distributed in the other (continuous phase); the suspended droplets may be surrounded by one or multiple layers. The droplets may be amorphous, liquid-crystalline, or any mixture thereof Microencapsulation systems may include emulsions, nanoemulsions, micelles, solid lipid nanoparticles, nanostructured lipid carriers, liposomes, nanoliposomes, niosomes, polymer particles, or hydrogel particles.

Microencapsulation techniques may include the emulsification and nanoemulsification techniques described herein, mixing, homogenization, injection, spray drying, spray cooling, spray chilling, freeze-drying, air suspension coating, fluidized-bed extrusion, centrifugal extrusion, coacervation, rotational suspension separation, cocrystallization, liposome entrapment, interfacial polymerization, molecular inclusion, microfluidization, ultrasonication, physical adsorption, complex formation, nanosized self-assembly, or any combination thereof. The microencapsulation process may be assisted or accelerated by the application of mechanical forces; such mechanical forces may include, but are not limited to the application of heat (e.g., through microwave irradiation), high shear/pressure forces (e.g., ultrasonic cavitation, homogenization), or mixing (e.g., using idealized chemical reactors, which may include, but are not limited to, batch reactors, continuous stirred-tank reactors, and plug flow reactors).

In one example, the cannabinoids can be dissolved in water-insoluble oil (i.e., carrier oil such as MCT) in the presence of emulsifiers so that the mixture can be formulated as an oil-in-water emulsion having a dispersed phase comprising oil/cannabinoid droplets, which can have micron- or nanometer-sized particle size distributions. It is understood that the bioavailability of cannabinoids can be improved by reduction in the droplet particle sizes, which increases surface area. The improved bioavailability is expected to enhance absorption of the cannabinoids. Accordingly, the cannabinoids of the present disclosure may be microencapsulated in an emulsion (i.e., hydrodynamic diameter>200 nm) or a nanoemulsion (i.e., hydrodynamic diameter<200 nm).

Preferably, the composition of the present disclosure comprises an oil-in-water emulsion and having a dispersed phase comprising oil droplets (comprising the at least one cannabinoid) with an average mean particle size of <200 nm, preferably <150 nm, preferably <100 nm, preferably <75 nm, preferably <70 nm, preferably <60 nm, or preferably <50 nm. An oil-in-water (O/W) emulsion has a dispersed phase comprising an organic material and a continuous phase that is water or an aqueous solution. This maximizes contact with the target mucosal membrane for transmucosal delivery.

The expression “average mean particle size” means the average particle size of all the particles of the dispersed phase (i.e. droplets). In some embodiments, the average particle size refers to D50, i.e., the particle diameter at the 50% point on a particle size distribution curve when the total volume is 100%. It is preferred that the emulsion is stabilized such that the oil droplets have the above prescribed average mean particle size ranges for at least the first week, preferably at least the first two weeks, preferably at least the first three weeks, or preferably at least the first four weeks up to 24 months after production.

It is important to note that it is particularly challenging to maintain small oil droplet particle size formed from the O/W emulsions containing the cannabinoids. Without wishing to be bound by theory, this is primarily due to an effect called Ostwald Ripening. Ostwald Ripening is the phenomena often found in oil-in-water emulsions in which smaller oil particles in solution spontaneously dissolve and deposit on larger oil particles to reach a more thermodynamically stable state wherein the surface area to volume ratio is minimized. The combination of destabilization by oil droplet collisions and coalescence, in addition to Ostwald Ripening in the case of volatile oils, can lead to the oil phase eventually becoming one big droplet to lower surface energy and minimize total surface area. As the droplets coalesce over time, the emulsion becomes unstable and eventually two separate phases. In an embodiment, Applicant has solved this formulation challenge by controlling the range of the average mean particle size of the oil phase droplets to those disclosed herein above by the incorporation of a viscosity modifier (as discussed below).

In another aspect, it is desirable to control the particle size distribution of the composition in order to minimize the discharged spray pattern of the composition in use. It has been found that greater variation in the particle size distribution of the droplets results in a significantly greater area covered by the spray action. This is undesirable since transmucosal administration, preferably intranasal and/or oral mucosa administration, relies on targeted delivery of the sprayed composition.

Therefore, it is preferable that the composition of the present disclosure comprises an oil-in-water emulsion comprising the cannabinoid, and wherein the emulsion comprises oil droplets having a D90 of <200 nm, preferably <150 nm, preferably <100 nm or preferably <50 nm. As used herein, the term “D90” means the particle size value, defined as the hydrodynamic diameter of the particles of the dispersed phase, corresponding to the cumulative size distribution at 90%, which represents the size of particles below which 90% of the sample lies. For example, a D90 of <200 nm means that 90% of the total amount of particles have a particle size of <200 nm.

In another embodiment, the composition of the present disclosure comprises an oil-in-water emulsion comprising the cannabinoid, and wherein the emulsion comprises oil droplets having a D50 of <200 nm, preferably <150 nm, preferably <100 nm or preferably <50 nm. As used herein, the term “D50” of <200 nm means that 50% of the total amount of particles have a particle size, defined as the hydrodynamic diameter of the particles of the dispersed phase, of <200 nm.

Particle size measurements can be performed using a system using Dynamic Light Scattering (DLS), for example, but not limited to the Zetasizer Nano (Malvern Panalytical). Dynamic Light Scattering (also known as “PCS-Photon Correlation Spectroscopy”) measures Brownian motion and relates this to the size of the particle. This is done by illuminating the particle with a laser and analyzing the intensity fluctuations in the scattered light. Details of the method are disclosed in U.S. Patent Publication No. 2013/0344120, which is incorporated herein in its entirety. The Zetasizer Nano System measures the rate of the intensity of the fluctuations and then uses this to calculate the size of the particles using mathematical algorithms.

“Particle size distribution” or “PSD” is an index (means of expression) indicating what sizes (particle size) of particles are present in what proportions (relative particle amount as a percentage where the total amount of particles is 100%) in the sample particle group to be measured. Volume, area, length, and quantity are used as standards (dimensions) for particle amount. However, generally, the volume standard is often used. The PSD is usually determined over a list of size ranges that covers nearly all the sizes present in the tested sample

Peak statistics are calculated using the expressions given below where Yᵢ is the Y value of the i^{th} Y axis class/bin and Xᵢ is the X axis value in the center of the X axis class/bin. The Y axis here is the Intensity (%) while the X axis is the diameter (nm). Area is defined as the area under each peak, relative to the total area of the distribution. Average mean particle size is defined as the average value of the peak, weighted by the Y axis parameter.

% Area=Σᵢ Yᵢ

Mean=pS(ᵢ)I(ᵢ)/Area

Polydispersity or Width of the Peak=Square root ((Σ_{Xi2Yi}/% area)−Mean²)

Polydispersity index (“PDI”) is a number calculated from a simple 2 parameter fit to the correlation data (the cummulants analysis). The PDI is dimensionless and scaled such that values smaller than 0.05 are seen with highly monodisperse standards. Values greater than 0.7 indicate that the sample has a very broad size distribution and is probably not suitable for the DLS technique. The various size distribution algorithms work with data that fall between these two extremes. The calculations for these parameters are defined in the ISO standard document 13321:1996 E and ISO 22412:2008.

The quantity of the oil phase in the oil-in-water emulsion can be from about 1 to about 50% on a (v/v) basis, preferably from about 5% to about 50% (v/v) and preferably from about 10% to about 20% (v/v)

It is desirable that the produced composition has at least 1 month, preferably at least 4 months, preferably at least 6 months, preferably at least 12 months, preferably at least 1 month to 24 months or longer, or preferably at least 24 months shelf-life and/or phase stability. By “phase stability”, it is meant that the emulsion formed from the cannabinoid-containing composition is stable against phase separation under storage conditions up to 40-50° C. for the specified amount of time.

### Viscosity Modifier

The compositions of the present disclosure comprise a viscosity modifier to provide a number of benefits such as, for example, desirable thermogelling of the composition, desirable rheology profile, desirable targeted spray pattern upon use, acceptable shelf-life stability, acceptable phase stability, and/or prevent acceleration of the oil droplets collisions and coalescence in the composition. In particular, the present disclosure relates to cannabinoid compositions having a rheology profile characterized by relatively high viscosity at about body temperature and relatively low viscosity at about room temperature.

The term “viscosity modifier” refers to an agent (a single agent or a combination of agents) that, upon dissolution or suspension in the liquid composition, causes the composition to undergo a change in physical properties after administration to a mucosa of a mammal. Such sites of application may include various administration routes, such as for example oral, vaginal, rectal, ocular, and nasal routes. The changes in physical properties of the composition may include: gelation of the liquid composition to result in the formation of a semi-solid or solid gel form and/or an increase in the viscosity of the composition. These changes in physical properties have the desired effect of enhancing or increasing contact/delivery of the composition, preferably the cannabinoid, to the mucosa of the mammal.

However, it is advantageous that changes in the viscosity of the composition are carefully controlled so that the changes in the physical properties of the composition occur at the appropriate time. For instance, it is undesirable for the composition to undergo this physical change prior to application to the mucosa. That is because if the formulation is too viscous, spray droplet formation may be hindered and the spray valve can become blocked. Alternatively, if the formulation is not viscous enough, excessive nebulisation may occur and a plume of cross section area may be formed. The resultant spray would no longer be targeted to the mucosa due to formulation pooling and could be swallowed (for the nasal and/or oral administration) or not entering the nasal passages (for nasal administration).

In some embodiments, simply adding any viscosity modifiers and/or any levels of the viscosity modifiers to the composition may not ensure the acceptable viscosity range needed to enhance transmucosal delivery of the composition while still avoiding the problems discussed above and/or the leakage problems. Preferably, the viscosity modifier has thermogelling properties, more preferably with reversible thermogelling properties

In some embodiments, the viscosity modifier is present in an amount effective to change the composition from a liquid (with lower viscosity) at about room temperature to a semi-solid or solid gel (with higher viscosity) when exposed to about body temperature. by “about room temperature”, it is meant from about 20° C. to 25° C.; by “about body temperature”, it is meant about 37° C. to 40° C. In some embodiments, the viscosity modifier operates to increase the viscosity of the composition when it is exposed to about body temperature, such that the composition containing the herein described components dispersed in an aqueous solvent forms a hydrogel. The transition between liquid and gel does not necessarily need to be at temperature to which the composition is exposed, but preferably the composition shall undergo transition in the interval between about 30° C. to about 37° C. In a preferred embodiment, the transition is sufficiently distinct at a defined temperature or at a fairly narrow temperature interval.

In some embodiments, the viscosity modifier is present in an amount effective to change the composition from a liquid (with lower viscosity) at about acidic pH below 6 to a semi-solid or solid gel (with higher viscosity) when exposed to about physiologic pH (around pH 7). In other words, the viscosity modifier operates to increase the viscosity of the composition when upon contacting a surface of a mucosa of the user. For example, Carbopol™ 971P increases viscosity of an aqueous composition upon a change in pH from 4 to 7, as described in the manufacturer technical data sheet (TDS-730, Ed. Aug. 13, 2010, Lubrizol).

In some embodiments, the composition has a viscosity at about room temperature such that the composition behaves like a liquid, is in a free-flowing state. For example, the composition may have an initial viscosity (e.g., at about room temperature or at non-physiological pH, such as storage pH) of less than about 2000 mPas, preferably less than 1000 mPas, preferably less than about 750 mPas, preferably less than 500 mPas, or preferably less than 200 mPas. In some embodiments, the composition has an applied viscosity upon contacting a surface of a mucose (e.g., upon exposure to about body temperature) such that the composition behaves like a semi-solid or solid gel. For example, the composition may have an applied viscosity of at least about 5000 mPas, such as at least about 7,500 mPas, at least about 10,000 mPas, at least about 15,000 mPas, at least about 20,000 mPas, at least about 25,000 mPas, at least about 30,000 mPas, at least about 40,000 mPas, at least about 50,000 mPas, or more. As used herein, the expression “initial viscosity” refers to the viscosity of the composition as measured at room temperature or at a non-physiological pH (e.g., storage pH). As used herein, the expression “applied viscosity” refers to the viscosity of the composition after it has been applied (e.g., sprayed) onto the mucosal surface. Typically, the “applied viscosity” is measured after the composition has transitioned to the gel form due to exposure to body temperature and/or change in pH. The viscosity of the compositions of the present disclosure can be measured using controlled-shear-rate rheometer, for example as measured with a rheometer with plate-plate geometry at a shear rate of 1 s⁻¹.

In one aspect, the viscosity modifier may be a poly(propylene oxide)/poly(ethylene oxide) copolymer. Preferably, the copolymer has a molecular weight of from about 7,000 g/mol to about 18,000 g/mol, and an ethylene oxide content of from about 30% to about 90% by weight of the copolymer. One preferred viscosity modifier is a block copolymer containing a polyoxyethylene block, i.e., a block made up of repeating ethylene oxide moieties, such as for example, a poly(propylene oxide)/poly(ethylene oxide) copolymer. A suitable viscosity modifier of this type is a Poloxamer or a mixture of more than one Poloxamer. See the Handbook of Pharmaceutical Excipients, 2^{nd} Edition Pharmaceutical Press, London, 1994, Eds, Wade and Weller.

In embodiments of the present disclosure, the block co-polymer may be a Poloxamer selected from the group consisting of: Poloxamer 108, Poloxamer 124, Poloxamer 182, Poloxamer 183, Poloxamer 184, Poloxamer 185, Poloxamer 188, Poloxamer 212, Poloxamer 217, Poloxamer 237, Poloxamer 217, Poloxamer 288, Poloxamer 331, Poloxamer 335, Poloxamer 338, Poloxamer 407, and a mixture thereof. In an embodiment, the Poloxamer comprise a mixture of the Poloxamer, such as for example, Poloxamer 407 and Poloxamer 188. In a non-limiting example, mixtures of Poloxamers include Poloxamer 188 and Poloxamer 407. Each of the Poloxamers may be present in equal amounts. Alternatively, each of the Poloxamers may be present in different amounts. For example, the weight ratio of different Poloxamers may be in the range of about 1:1 to about 1:10, or any weight ration therebetween; in another example, the weight ratio of different Poloxamers may be of at least about 1:6. In a specific, non-limiting example, the viscosity modifier may be a mixture of Poloxamer 188 and Poloxamer 407 in a weight ratio of 0.1:5.3 or higher; alternatively, the mixture of Poloxamer 188 and Poloxamer 407 comprises at least 85% Poloxamer 407.

In one aspect, the viscosity modifier may be present in an amount effective to change the composition from a liquid at about room temperature to a semi-solid or solid gel upon exposure to about body temperature. In this context, the term “effective” means an amount of the viscosity modifier sufficient to allow for the thermogelling of the composition. An effective amount of the viscosity modifier will vary broadly with the particular composition, the type of modifier used, and the like factors. Preferably, the viscosity modifier is present in an amount of from about 0.1 wt. % to about 10 wt. %, from about 0.3 wt. % to about 8 wt. %, from about 0.5 wt. % to about 7 wt. % or from about 1 wt. % to about 7 wt. %, by weight of the composition.

In embodiments of the present disclosure, cannabinoid component and the viscosity modifier are present in a ratio (w/w) of cannabinoid component:viscosity modifier within 0.9-1.1:1-9.5 or any ratio within this range. The amount of viscosity modifier (in wt. %) required may vary based on the amount of cannabinoid component (in wt. %) in the composition. More specifically, the (w/w) ratio of cannabinoid component:viscosity modifier may vary from 0.9-1.1:7.9-9.5 when the cannabinoid component is about 1 wt. %, or from 0.9-1.1:3.2-3.5 when the cannabinoid component is about 3 wt. %, or from 0.9-1.1:1-1.2 when the cannabinoid component is about 5 wt. %.

In another aspect, the compositions of the present disclosure increase in viscosity upon contact with the mucosa of a mammal. Preferably, the viscosity increases by at least 50%, preferably at least 60%, preferably at least 70%, preferably at least 80%, preferably at least 90% or preferably at least 100% (relative to the viscosity prior to the administration).

The change in viscosity of the composition upon contact with the mucosa may be determined by measuring the viscosity of the composition under simulated physiological conditions. For example, the viscosity of a given composition may be measured in simulated conditions in the oral cavity such as the temperature of the gums or the internal cheeks.

Due to the physical changes described above, the composition of the present disclosure has a longer residence time on the mucosal surfaces, thereby increasing the topical contact of the composition, preferably the cannabinoid, on the mucosal surfaces. In one aspect, the compositions of the present disclosure have increased residence time on the nasal and/or oral mucosa. Preferably, the composition exhibits a mucosal surface residence time of greater than about 20 seconds, preferably greater than about 30 seconds, preferably greater than about 40 seconds, preferably greater than about 50 seconds, preferably greater than about 1 minute, preferably greater than about 2 minutes, preferably greater than about 5 minutes, preferably greater than about 10 minutes, preferably greater than about 20 minutes following application on the surface of the mucosa. In the case of sublingual or buccal delivery, this means there is reduced chance that the administered cannabinoids will be swallowed by the subject and that more cannabinoids will be absorbed transmucosally.

### Adjunct Components

The composition of the present disclosure may optionally comprise a number of other adjunct components such as flavorants, permeation enhancers, preservatives, antioxidants, surfactants, bulking agents, colorants, pH modifiers, sweeteners, mucoadhesive agents, taste-masking agents, or the like. The amount of each of these components in a given composition will be optimized for each formulation, to obtain a stable product (dosage form) having the desired shelf-life.

It is recognized that adjunct components may perform more than one function and are therefore characterized as having different uses depending on the specific application. While the use of a component in the context of a particular formulation may determine the function of the component, the inclusion of any particular component into any one or more category as set forth below is not meant to limit the function of that component.

### Flavorant

It is desirable that the compositions of the present disclosure, particularly if intended for oral administration, do not have a disagreeable taste. As used herein, the term “taste” means a perception arising from the interaction of the composition with taste receptors in the mouth. In the context of the present disclosure, a disagreeable taste can be a taste that is perceived such as, for example but not limited to, unpleasant, sharp, bitter, or synthetic. For example, the composition may contain an amount of emulsifiers, common in aqueous formulations containing forms of cannabinoids, that results in a perceived synthetic or bitter taste.

Accordingly, the composition may contain an effective amount of a flavorant to mask the disagreeable taste. The composition herein may include from about 0.01 wt. % to about 5 wt. %, preferably from about 0.01 wt. % to about 4 wt. %, preferably from about 0.1 wt. % to about 3 wt. %, preferably from about 0.5 wt. % to about 2 wt. %, or a combination thereof of a flavorant, by weight of the composition. Any suitable flavorant known in the art may be used in the present compositions.

Generally suitable flavorants, also referred to herein as “flavor ingredients”, are chemicals with structural features and functional groups that are less prone to redox reactions. These include derivatives of flavor ingredients that are saturated or contain stable aromatic rings or ester groups. In some embodiments, the flavorant may be an extract of a natural ingredient or foodstuff, or may be a terpene.

In a non-limiting example, the composition may comprise a flavorant selected from the group consisting of extracts of cinnamon, cucumber, mint, orange, lime, citrus, cookie dough, chocolate, vanilla, jasmine, lychee, almond, banana, grape, pear, pineapple, pine, oak, apple, pumpkin, grapefruit, watermelon, cotton sugar, durian, longan, taro, sapote, toffee nut, caramel, lotus, mango, mangosteen, coconut, coffee, strawberry, passion fruit, blueberry, raspberry, kiwi, walnut, cocoa, cherimoya, custard apple, papaya, fig, plum, nectarine, peaches, guava, honeydew, jackfruit, kumquat, loquat, palm, pomelo, persimmon, quince, cassia, sage, marjoram, lemon, orange, tamarind, and combinations thereof.

Other examples of suitable flavorants include, but are not limited to, mint oils, wintergreen oil, clove bud oil, parsley oil, propenyl guaethol, heliotropine, 4-cis-heptenal, diacetyl, methyl-p-tert-butyl phenyl acetate, methyl salicylate, ethyl salicylate, 1-menthyl acetate, oxanone, a-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, a-terpineol, linalool, limonene, citral, neral, geranial, geraniol nerol, maltol, ethyl maltol, anethole, dihydroanethole, carvone, menthone, beta-damascenone, ionone, gamma-decalactone, gamma-nonalactone, y-undecalactone, or combinations thereof.

### Permeation Enhancer

Membrane permeability is the limiting feature for mucosal delivery. Epithelium that lines the mucosa is a very effective obstacle to the permeation and hence absorption of the cannabinoids. Permeation enhancers (which may also be referred to as “penetration enhancers”, “absorption enhancers” or “permeability enhancers”) may be used to facilitate the permeation through the mucosa. The composition may comprise one or more permeation enhancer in an amount of from about 0.5 wt. % to about 15 wt. %, preferably about 1.0 wt. % to about 10 wt. %, preferably from about 1 wt. % to about 5 wt. %.

Suitable examples of permeation enhancers are selected from the group consisting of: chelators (e.g., EDTA, EGTA, citric acid, salicylates, N-acyl derivatives of collagen, and enamines (N-amino acyl derivatives of 3-diketones)), surfactants (e.g., sodium lauryl sulfate, polyoxyethylene-9-laurylether and polyoxyethylene-20-cetylether; and natural surfactant such as bile salts (sodium deoxycholate, sodium glycocholate and sodium taurocholate)), fatty acids and derivatives thereof (e.g., sodium caprylate, sodium caprate, sodium laurate, lauric acid, oleic acid, lecithin, phospholipids >60% (phosphatidylcholine, phosphatidylethanolamine, phosphatidylinositol, phosphatidic acid) monoolein and acylcarnitines)), terpenes (e.g., I-menthol, β-caryophyllene, limonene, myrcene, or α-pinene), cyclodextrins, azone, chitosan, and lysalbinic acid. Other suitable examples of permeation enhancers may be found in at least U.S. Pat. No. 6,328,992; and PCT Publication WO 2003/101357, which are herein incorporated by reference in their entirety. Other suitable examples of permeation enhancers may include hyaluronic acid (HA) (also known as hyaluronan or hyaluronate) or base. The HA may have a molecular weight in the range of from about 5 to 20,000 kDa. Preferably, the HA has a molecular weight of 50 kDa to 2,000 kDa (e.g., 70 kDa to 1,500 kDa, 200 kDa to 1,500 kDa, 500 kDa to 1,500 kDa, or 700 kDa to 1,500 kDa).

### Preservative

The composition of the present disclosure may include one or more preservative. Preservatives include compounds used to prevent the growth of microorganisms. Suitable preservatives include, but are not limited to, ethyl paraben, propyl paraben, benzyl alcohol, benzoic acid, sodium benzoate, sorbic acid, phenol, phenylethyl alcohol, phenylmercuric nitrate and thimerosal and others known to those of ordinary skill in the art. If the composition contains a preservative, the composition preferably comprises from about 0.05 wt. % to about 2 wt. %.

### Antioxidant

The composition of the present disclosure may include one or more antioxidant. As used herein, the term “antioxidant” is intended to mean an agent that inhibits oxidation and thus is used to prevent the deterioration of preparations by oxidation. Suitable examples of antioxidants include, but are not limited to, ascorbic acid, ascorbyl palmitate, butylated hydroxyanisole (BHA), butylated hydroxytoluene (BHT), hypophophorous acid, monothioglycerol, sodium ascorbate, sodium formaldehyde sulfoxylate, and sodium metabisulfate and others known to those of ordinary skill in the art. Other suitable antioxidants include, for example, vitamin C, sodium bisulfite, vitamin E and its derivatives, propyl gallate, a sulfite derivative, and others known to those of ordinary skill in the art. If the composition contains an antioxidant, the composition preferably comprises from about 0.01 wt. % to about 5 wt. %.

### Surfactant

The composition of the present disclosure may include at least one surfactant to assist in emulsifying the cannabinoid.

Suitable examples of surfactants include, but are not limited to, Tweens (polysorbates) such as Tween™ 20 (polyoxyethylene sorbitan monolaurate), Tween 40 (polyoxyethylene sorbitan monopalmitate), Tween 60 (polyoxyethylene sorbitan monostearate), and Tween 80 (polyoxyethylene sorbitan monooleate), sugar esters such as sucrose monopalmitate, sucrose monostearate, sucrose distearate, sucrose polystearate, quillaja saponin (for example, but not limited to Q-Naturale®) and components thereof, sorbitan esters (Spans) such as Span™ 20 (sorbitan monolaurate), Span 40 (sorbitan monopalmitate), Span 60 (sorbitan monostearate), Span 80 (sorbitan monooleate), or any mixtures thereof.

Additional examples of suitable surfactants include, but are not limited to, Capryol 90, Cremophor™ RH40, Gelucire™ 44/14, Gelucire 50/13, Imwitor™ 91, Imwitor 308, Imwitor 380, Imwitor 742, Imwitor 780K, Imwitor 928, Imwitor 988, Labrafil™ M 1944 CS, Labrafil M 2125 CS, Lauroglycol™ 90, Tagat™ TO, or any mixtures thereof.

Additional examples of suitable surfactants include, but are not limited to, PEG-2 Hydrogenated Caster Oil, Sorbitantrioleate, Sorbitantristearate, Sorbitan Esters, Glycerol Stearate, Sorbitan Sesquioleate, Sorbitan Oleate, Sorbitan Monostearate, PEG-2 Oleyl Ether, PEG-2 Stearyl Ether, PEG-7 Hydrogenated Caster oil, PEG-2 Cetyl Ether, PEG-4 Sorbitan Stearate, PEG-2 Sorbitan Isostearate, Sorbitan Palmitate, Phosphatidylcholine, Sorbitan Monolaurate, PEG-40 Sorbitan Peroleate, PEG-4 Lauryl Ether, Polysorbate 81, PEG-40 Sorbitan Hexaoleate, PEG-40 Sorbitan Perisostearate, PEG-10 Olive Glycerides, PEG sorbitol Hexaoleate, Polysorbate 65, Methylcellulose, Gum Arabic, Captex 300, PEG-7 Glyceryl Cocoate, PEG-8 Stearate, PEG-400 Monoleate, PEG-400 Monostearate, Sugar Ester Stearate (S-1170, S-1570, S-1670), PEG-15 Glyceryl Isostearate, PEG-35 Almond Glycerides, PEG-10 Oleyl Ether, PEG-8 Isooctylphenyl Ether, PEG-10 Stearyl Ether, PEG-35 Caster Oil, Nonoxynol-9, PEG-10 Cetyl Ether, PEG-400 Monolaurate, Q-Naturale 200 (Quilaja extract), PEG-40 Hydrogenated Caster Oil, PEG-12 Tridecyl Ether, PEG-18 Tridecyl Ether, Polysorbate 60, Polysorbate 80, PEG-20 Glycerol Stearate, Sugar Ester Oleate (OWA-1570), PEG-20 stearylether, Polysorbate 40, Sugar Ester Palmitate (P-1570, P-1670), Sugar Ester Laurate (LWA-1570, L-1695), Polysorbate 20, PEG-60 Hydrogenated Caster Oil, Tocopherol Polyethylene Glycol Succinate (TPGS), PEG-40 Stearate, PEG-50 Stearate, Purity Gum™ Ultra, Purity Gum BE, or any mixtures thereof In one non-limiting example, the surfactant may be a polyethylene glycol derivative of castor oil, for example a PEG40 hydrogenated castor oil.

In certain embodiments, when surfactant is included in the formulation, the surfactant is included in an amount of from about 0.01% to about 15%, preferably 1% to 15%, preferably 5% to 15%, or preferably 10% to 15% by weight of the composition.

In embodiments of the present disclosure, the cannabinoid component and the at least one surfactant are present in a ratio (w/w) of cannabinoid component: surfactant within 0.9-1.1:1.9-2.2, or any ratio within this range. The amount of surfactant (in wt. %) required may vary based on at least the amount of cannabinoid component (in wt. %) and/or viscosity modifier (in wt. %) in the composition. In a non-limiting example, the (w/w) ratio of cannabinoid component:surfactant may be within 0.9-1.1:1.4-1.9 when the cannabinoid component is about 1 wt. %, or may be within 0.9-1.1:1.8-2.2 when the cannabinoid component is about 3 wt. %, or may be within 0.9-1.1:1.9-2.2 when the cannabinoid component is about 5 wt. %.

### Bulking Agent

The composition of the present disclosure may include one or more bulking agents. Bulking agents may also be used in accordance with certain embodiments of the present disclosure including for example, but not limited to, microcrystalline cellulose, mannitol, xylitol, starches and the like. Preferably, the bulking agent is mannitol. In certain preferred embodiments wherein bulking agent is included in the formulation, the bulking agent is included in an amount of from about 0.001% to about 10%, preferably from about 0.01% to about 5%, by weight of the composition.

### Colorant

The composition of the present disclosure may include one or more colorants. As used herein, the term “colorant” is intended to mean a compound used to impart color to liquid compositions. The colorant may be in the form of pigments, dyes, or opacifiers. The colorant may be in the form of an aqueous solution, preferably 1% colorant in a solution of water.

Suitable examples of colorants include, but are not limited to, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide red. Other suitable colorants include titanium dioxide and natural coloring agents such as grape extract, beet red powder, carmine, turmeric, paprika, and others known to those of ordinary skill in the art. It will be appreciated that selected components for the compositions must be chemically and physically compatible with one another.

### pH Modifiers

The composition of the present disclosure may include one or more pH-adjusting agents to improve stability and/or solubility. It is believed that the pH modifiers can also control cannabinoid release and enhance bioadhesion of the composition to the mucosa. Preferably, the pH of the composition is from about 5 to about 9, preferably from about 5.5 to about 7.5, preferably from about 6.0 to about 7.0. Alternatively, the pH can be greater than 6, alternatively greater than 7, alternatively from 8 to 10, or combinations thereof.

The pH of the composition may be modified using any pharmaceutically acceptable means. Suitable examples of pH modifiers include, but are not limited to, organic acid or base, preferably tartaric acid, phosphoric acid, hydrochloric acid, maleic acid, sodium hydroxide, citric acid and the like known to those of ordinary skill in the art.

### Sweetener

The composition of the present disclosure may include one or more sweetening agent (which is different from a flavorant). If the composition contains a sweetener, the composition preferably comprises from about 0.005% to about 5%, alternatively from about 0.01% to about 1%, by weight of the composition, alternatively from about 0.1% to about 0.5%, by weight of the composition, or alternatively combinations thereof. Suitable examples of sweeteners include, but are not limited to, sucralose, sucrose, aspartame, saccharin, dextrose, mannitol, xylitol, or a combination thereof.

Mucoadhesive agent

The composition of the present disclosure may include one or more mucoadhesive agent, such as natural, semisynthetic, synthetic polymers or combinations thereof. Suitable natural polymers and semi-synthetic polymers may include, but are not limited to, amylopectin, zein, modified zein, casein, gelatin, pectin, agar, serum albumin, collagen, chitosan, oligosaccharides and polysaccharides such as pyrrolidones, dextrins, cellulose and cellulose compounds (such as methylcellulose, carboxymethyl cellulose, hydroxyethyl cellulose, hypromellose), dextrans, tamarind seed polysaccharide, gellan, carrageenan gum, xanthan gum, Arabic gum, guar gum, tragacanth gum, hyaluronic acid, polyhyaluronic acid, alginic acid or sodium alginate, locust bean gum, pullulan, and the like. It is to be noted that any of the herein listed compound can also include corresponding salts thereof, e.g., hyaluronic acids and salts thereof, alginic acids and salts thereof, carrageenans and salts thereof, and the like. Suitable synthetic polymers include polycarbophil, Noveon AA-1, Carbopol compounds, and Carbomer compounds.

In addition to the adjunct components listed above, the present compositions can comprise the usual and conventional ancillary components that are known to one skilled in the art. It will be appreciated that selected components for the compositions must be chemically and physically compatible with one another.

### Cannabinoid Products

The present disclosure is also directed to a cannabinoid product for administration to a mucosa of a subject, wherein the product comprises the cannabinoid composition according to the present disclosure and, optionally, may include one or more pharmaceutically acceptable excipients.

As used herein, the term “pharmaceutically acceptable excipient” means a suitable vehicle or ingredient, which can be used to form and/or apply the present composition to the mucosa surface in a safe and effective manner according to established governmental standards. Suitable pharmaceutical acceptable excipients and their formulations are described in “Remington's Pharmaceutical Sciences” by E. W. Martin, 1975, Mack Publishing Co.

In particular, suitable examples may include liquid carrier, such as for example, water, ethanol, saline, aqueous dextrose, glycols, oils (including those of petroleum, animal, vegetable or synthetic origin) and the like, or a mixture thereof. Preferably the excipient is water, preferably USP water, due to its many benefits. For example, water is useful as a processing aid and is benign to the nasal and/or oral cavity. Water may be added as an ingredient in its own right or it may be present as a carrier in other common raw materials. The water content as used herein means the total amount of water present in the thermogelling cannabinoid composition, whether added separately or as a solvent or carrier for other raw materials.

Preferably, the pharmaceutically acceptable excipient may be present at a level of from about 40 wt. % to about 80 wt. %, preferably about 50 wt. % to about 80 wt. %, or any amount therebetween; for example, the pharmaceutically acceptable excipient may be present at a level of about 50 wt. %, 60 wt. %, 70 wt. %, or 80 wt. %. In some embodiments, the pharmaceutically acceptable excipient may be present at a level of at least 50 wt. % by weight of the composition; for example, and without wishing to be limiting, the pharmaceutically acceptable excipient may be present at a level of about 70 wt %.

The compositions for use in the present disclosure may take any form suitable for mucosal administration. These include, but are not limited to, vapor sprays, aerosols, emulsions, liquids and the like.

It is particularly preferred that the compositions of the present disclosure may be included in an article of manufacture comprising a spray dispenser. As a result, it is desirable that the composition of the present disclosure can be formulated for delivery to, for example, the nasal and/or oral cavity, using spray devices known in the art such as for example, via a pump action or pressurized administration vessel such as an aerosol spray (e.g., compressed air-, jet-, ultrasonic-, and piezoelectric nebulizers available from Pfeiffer and Valois). Such devices are familiar to the skilled artisan and can provide metered doses of the compositions, such as for example, single or multiple dosing, as desired.

Preferably, the formulation of the composition is such that it provides a fine micellized mist spray comprising the cannabinoid suitable for mucosal delivery for effective transdermal absorption across the mucosa. Without wishing to be bound by theory, the fine mist ensures maximum surface coverage and therefore optimum delivery via transmucosal delivery, for example via the nasal and/or oral mucosa.

In an embodiment, the product is an oral spray form, preferably a sublingual liquid spray form and/or a buccal liquid spray form. The oral route of administration is attractive because it is a non-invasive route of administration, with the advantage of avoiding the first-pass metabolism, sustained action and ease of use. The sublingual mucosa and buccal mucosa are preferred due to the fact that permeability is greater there and it is possible to realize transmucosal effect (i.e., systemic effect) of the cannabinoid administration.

In another embodiment, the product is an intranasal spray form, preferably an intranasal liquid spray form. The intranasal route of administration is also desirable because the nasal mucosa is rather porous and thin endothelial basal membrane. The nasal mucosa also has rapid blood flow, with a highly vascularized epithelial layer for relatively fast transcellular absorption of the administered cannabinoids. Similar to the oral route of administration, the intranasal route of administration has the advantages of bypassing first pass effect, avoiding presystemic metabolism and achieving rapid systemic blood levels.

Typically, the compositions intended for intranasal administration are in an aqueous solution, such as for example, a nasal liquid spray form. Such formulations may be conveniently prepared by dissolving compositions according to the present disclosure in water, or as an oil-in-water emulsion, to produce an aqueous solution, and rendering the solution sterile. Suitable examples of systems for dispensing liquids as a nasal spray as disclosed in at least U.S. Pat. Nos. 4,511,069; 4,778,810; 6,080,762; 7,052,678; and 8,277,781, each of which is hereby incorporated by reference in its entirety.

In another aspect, the present disclosure also relates to improving the physiological condition and/or health of animals, preferably companion animals (e.g., pets). Similar to humans, it is desirable to enable animals to have a feeling of physical and/or emotional satisfaction. Alternatively, animals may also suffer from a disease or condition, particularly anxiety, pain and inflammation, which can be crippling for them. For most pet owners watching their pets suffer unnecessarily with these symptoms can be difficult. Therefore, there is a need for a product to improve the physiological condition and/or health of animals. In some embodiments, the product may be useful in treating a disease or condition in an animal. More specifically, the present disclosure is directed to a product in the form of a spray, useful for improving the physiological condition and/or health of the animals.

It will be understood that the products according to the present disclosure comprise cannabinoids at levels that are in compliance with regulations on cannabis products in various countries, such as for example, the Health Canada Proposed Regulations for Additional Cannabis Products as published on Dec. 20, 2018 (www.canada.ca/en/health-canada/services/drugs-medication/cannabis/resources/proposed -regulations-edible-cannabis-extracts-topicals.html). For example, when the product includes THC, it is preferred that the product comprises a maximum amount of about 20 mg or less, preferably about 15 mg or less, preferably about 10 mg or less or preferably about 5 mg or less of the cannabinoid, including THC and THCA, in a package unit.

### Method, Use and Kits

The present disclosure relates to methods for producing a desirable physiological effect in a subject. Preferably the desired effect is associated with a feeling of physical and/or emotional satisfaction in the subject. Alternatively, the present disclosure also relates to methods for treating a disease or condition in a subject. The methods according to the present disclosure comprise administering to a mucosa, preferably a nasal and/or oral mucosa, of the subject an effective amount of the composition according to the present disclosure. In an embodiment of the method, whereby after the administration step the cannabinoid composition forms a gel on a mucosa thereby increasing a residence time of the gel on the mucosa. As previously discussed, this transformation is clearly beneficial to enhance bioavailability of the administered cannabinoid on the mucosa for systemic absorption.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a subject. As used herein, the term “subject” to which a composition can be administered include mammals such as for example, a mammal, human, companion animals (e.g., cats and dogs), farm animals (e.g., cattle, goats, sheep, horses, pigs and chickens), performance animals (e.g., race horses) and laboratory test animals (e.g., rats) Typically, the subject is a human.

In certain embodiments, the disease or condition is selected from the group consisting of:

pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction. In the context of these methods, it is preferable that the cannabinoid composition provides substantially no psychoactive effect or no psychoactive effect.

In the context of recreational use, the “effective amount” administered and rate and time-course of administration, will depend on the desired effect associated with a feeling of physical and/or emotional satisfaction in the subject.

In the context of health and wellness, the “effective amount” administered and rate and time-course of administration will depend on the nature and severity of the disease or condition being treated and typically also takes into consideration the condition of the individual subject, the method of administration and the like.

Preferably, an effective amount of the composition, typically from about 0.05 mL to about 2 mL is administered to the mucosal surface. The composition may be administered applied utilizing a spray apparatus, such as for example, a vaporizer or atomizer. The scope of the present disclosure should be considered to cover one or more distinct applications of the composition or the continuous release of a composition a vaporizer or other type of atomizer. The composition may be administered via nasal or oral (e.g., buccal administration or sublingual administration), vaginal, ocular or rectal administration route. Preferably, the composition is administered nasally, orally or both.

The present disclosure further relates to a kit comprising the composition of the present disclosure and instructions for use.

### EXAMPLES

The following examples describe some exemplary modes of making and practicing certain compositions that are described herein. It should be understood that these examples are for illustrative purposes only and are not meant to limit the scope of the compositions and methods described herein.

### Example 1

### Thermogelling Composition Preparation

In this example, thermogelling compositions were prepared. The compositions were formulated as nanoemulsions.

For each formulation (F1-F16) shown below, the following steps were performed.
(1) The water phase ingredients (water, Poloxamer) were solubilized with stirring.
(2) Separately, the oil phase ingredients (ethanol, THC distillate, PEG-40 castor oil, and MCT oil) were solubilized with stirring.
(3) Once prepared, the respective water and oil phases were combined by slow addition of the oil phase to the water phase, with constant stirring by magnetic agitation at ˜500 rpm.

Compositions comprising varying amounts of THC distillate (1 wt. %, 3 wt. %, 5 wt. %) were prepared as described above; representative formulations are shown in Tables 1 to 3 (other data not shown). In each case, the distillate used contained approximately 90% THC.

TABLE 1
Composition Formulations - 1 wt. % Distillate. The amount
of each component in each of the formulations is shown
(in wt. %). Polo407 = Poloxamer 407; Polo188 =
Poloxamer 188; EtOH = 94% ethanol; PEG-40 =
PEG40 Hydrogenated castor oil; THC = THC distillate;
MCT = MCT oil. A dash indicates that the component was not added.

| Formulation | Water | Polo407 | Polo188 | EtOH | PEG-40 | THC | MCT |
|---|---|---|---|---|---|---|---|
| F1 | 73.8 | 15.68 | 0.7 | 7.4 | 1.4 | 1.1 | — |
| F2 | 77.2 | 13.72 | 0.6 | 6.4 | 1.2 | 0.9 | — |
| F3 | 76.5 | 11.32 | 0.5 | 8.7 | 1.7 | 1.2 | — |
| F4 | 79 | 9.13 | 0.4 | 8.5 | 1.7 | 1.2 | — |
| F5 | 79.5 | 9.18 | 0.4 | 8.6 | 1.2 | 1.2 | — |
| F6 | 78.5 | 9.07 | 0.4 | 8.5 | 2.3 | 1.2 | — |

TABLE 2
Composition Formulations - 3 wt. % Distillate. The amount
of each component in each of the formulations is shown
(in wt. %). Polo407 = Poloxamer 407; Polo188 =
Poloxamer 188; EtOH = 94% ethanol; PEG-40 =
PEG40 Hydrogenated castor oil; THC = THC distillate;
MCT = MCT oil. A dash indicates that the component was not added.

| Formulation | Water | Polo407 | Polo188 | EtOH | PEG-40 | THC | MCT |
|---|---|---|---|---|---|---|---|
| F8 | 77.6 | 8.96 | 0.4 | 4.4 | 5.8 | 2.9 | |
| F9 | 79.9 | 9.23 | 0.4 | 4.5 | 3.0 | 3.0 | — |
| F10 | 78.8 | 9.10 | 0.4 | 4.4 | 4.4 | 2.9 | — |

TABLE 3
Composition Formulations - 5 wt. % Distillate. The amount
of each component in each of the formulations is shown
(in wt. %). Polo407 = Poloxamer 407; Polo188 =
Poloxamer 188; EtOH = 94% ethanol; PEG-40 =
PEG40 Hydrogenated castor oil; THC = THC distillate;
MCT = MCT oil. A dash indicates that the component was not added.

| Formulation | Water | Polo407 | Polo188 | EtOH | PEG-40 | THC | MCT |
|---|---|---|---|---|---|---|---|
| F11 | 67.4 | 7.79 | 0.3 | 8.2 | 10.8 | 5.4 | — |
| F12 | 68.9 | 5.89 | 0.3 | 8.4 | 11.1 | 5.5 | — |
| F13 | 75.1 | 5.31 | 0.3 | 3.7 | 10.1 | 5.5 | — |
| F14 | 69.2 | 5.75 | — | 8.3 | 11.1 | 5.6 | — |
| F15 | 69.25 | 5.75 | — | 8.33 | 11.11 | — | 5.55 |
| F16 | 69.25 | 5.75 | — | 8.33 | 11.11 | 5.55 | — |

See Example 2 for discussion of compositions and results.

### Example 2

### Thermogelling Composition Characterization

In this example, the particle size of the thermogelling compositions prepared in Example 1 was measured.

Particle Size Measurement—The particle size for compositions of Example 1 was measured in water solution at 25° C. using dynamic light scattering (DLS). All samples in the present disclosure have been analyzed at a dilution of 1/20 in purified water using a LiteSizer™ (Anton Paar GmbH, Germany). Particle size measurement were performed again for composition F16 using the same method after two months of storage at 25° C. Results of particle size measurements are shown in Table 4.

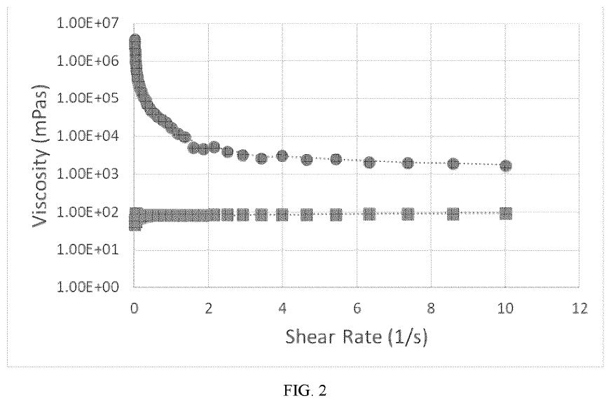

Gelation Point Measurement—The gelation point of compositions of Example 1 was measured using an MCR 92 Rheometer from Anton Paar. The storage and loss moduli were measured at a constant shear rate of 1 s⁻¹, and an increase in temperature of 1° C. per minute using a PP50 geometry. The crossover point between the storage modulus and the loss modulus on the resulting gelation curve yields the gelation temperature of the composition. A representative gelation curve is shown in FIG. 2 (for composition F6) and gelation temperatures are shown in Table 4.

TABLE 4
Particle Size and Gelation Point of Compositions. The dispersed
phase particle size (nm) and the gelation temperatures (°
C.) for formulations of Tables 1 to 3 are shown. A dash (—) indicates
that the characteristic was not measured.

| Formulation | Particle Size (nm) | Gelation Temperature (° C.) |
|---|---|---|
| F1 | — | 25 |
| F2 | — | 27 |
| F3 | — | 29 |
| F4 | 53 | 31.5 |
| F5 | 117 | 33 |
| F6 | 39 | 34 |
| F7 | 42 | 32 |
| F8 | 42 | 31-32 |
| F9 | 280 | |
| F10 | 150 | 33 |
| F11 | 42 | 25 |
| F12 | 52 | 31 |
| F13 | 80 | 34 |
| F14 | 41 | 32 |
| F15 | 42 | — |
| F16 | 42 | — |
| F16 * | 44 | — |

* after two months of storage at 25° C.

Viscosity Measurement—Following preparation of the composition, the viscosity of composition F14, prepared in Example 1, was measured using a MCR92 rheometer (Anton Paar) with plate-plate geometry. The tests were performed at temperatures of 25° C., 40° C., and a ramp from 20° C.-40° C. The shear stress and apparent viscosity data were obtained in a controlled rate method ranging from 0.01 to 10% shear rate, or at a fixed rate of 1% shear rate. All analyses were carried out in duplicates. Results of viscosity measurements are shown in Table 5.

TABLE 5
Viscosity of Compositions. The viscosity (in mPas) for
composition F14 is shown. Values for viscosity are shown
at 25° C. and at 40° C.

Viscosity

| Formulation | 25° C. | 40° C. |
|---|---|---|
| F14 | 80-90 mPas | 10,000-20,000 mPas |

The compositions prepared in Example 1 are self-emulsifying nanoemulsion compositions. The compositions were developed iteratively. Beginning with compositions comprising 1 wt. % THC distillate then moving to 3 wt. % and 5 wt. % THC distillate and using particle size and gelation temperatures as a guide. The desired gelation temperature for the compositions was above 30° C., and the desired particle size was less than 55 nm.

It was determined through experimentation (data not shown) that the viscosity modifiers must be dissolved in water prior to nanoemulsion formation, otherwise the particle size of the nanoemulsion increased. Initial formulations (F1 to F3) served to determine the amounts of component (viscosity modifier (e.g., Poloxamer)) needed to achieve a gelation temperature near body temperature. Additionally, it was determined experimentally that the THC distillate could be replaced in whole or in part by MCT oil, with no significant effect on particle size (see results for compositions F15 and F16). Therefore, it appears that MCT oil as a carrier is not a required component in the compositions. In view of this interchangeability, the distillate and MCT oil are together referred to as the “cannabinoid component”.

An overall optimal ratio of cannabinoid component:viscosity modifier:surfactant in the compositions of this example is about 0.9-1.1:1-9.5:1.9-2.2 was observed over all experimental results. Generally, as the amount of Poloxamer in the composition decreased, the amount of PEG40 hydrogenated castor oil was increased in order to maintain the desired gelation temperature and particle size—see for example F4 to F6. It also appears that the optimal ratio of cannabinoid component:viscosity modifier:surfactant varies based on different loading of the cannabinoid component.

For example, in compositions loaded with ˜1 wt. % THC distillate, having a ratio of 0.9-1.1:7.9-9.5: 1.4-1.9 (cannabinoid component:Poloxamer:surfactant) afforded a particle size <55 nm and gelation temperature of >30° C. In compositions loaded with ˜3 wt. % THC distillate, a cannabinoid component:viscosity modifier:surfactant ratio of 0.9-1.1:3.2-3.5:1.8-2.2 also afforded a particle size <55 nm and gelation temperature of >30° C. In compositions loaded with ˜5 wt. % THC distillate, a cannabinoid component:viscosity modifier:surfactant ratio of 0.9-1.1:1-1.2:1.9-2.2 also afforded a particle size <55 nm and gelation temperature of >30 ° C..

It was also observed that a portion of the Poloxamer 407 (0 to 0.75 wt. %) could be replaced with Poloxamer 188 with no effect on particle size, but with a corresponding increase in the temperature at which gelation occurs (data not shown). Finally, the F16 composition showed stability of the particle size in the compositions for a period of 2 months.

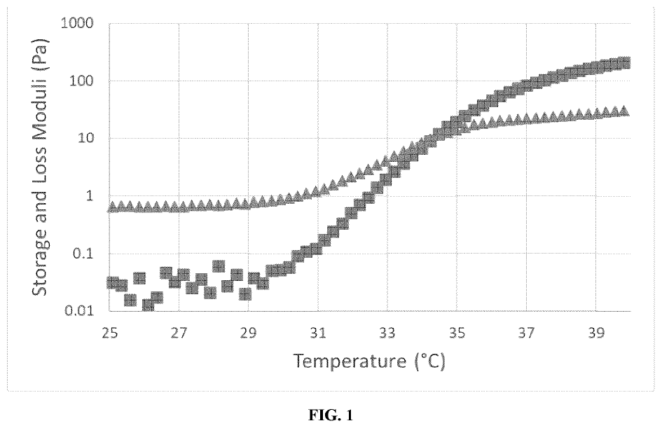

For compositions comprising of 5% cannabinoid component, using 1:1:2 ratio of THC distillate:Poloxamer 407:PEG40 hydrogenated castor oil (F12, F14), the increase in the composition's viscosity (i.e., gelation) occurs at 31 to 33° C. A representative gelation curve (composition not shown) is shown in FIG. 1, where the gelation temperature was 34° C. As shown in F11-F16, varying the amount of Poloxamer 407 in the composition, within the optimal 0.9-1.1:1-1.2:1.9-2.2 cannabinoid component:viscosity modifier:surfactant ratio, causes gelation to occur at a varying temperatures, though no significant effect on particle size is noted.

FIG. 2 shows that the viscosity of the composition F14 was found to be shear independent at 25° C. and was measured to be 80-85 mPas; however, the viscosity was found to be shear thinning at 40° C., with a value of 10,000-20,000 mPas at low shear (1% shear rate).

### Example 3

### Thermogelling Composition Containing Other Ingredients

In this example, compositions comprising mucoadhesive agents or flavorants were prepared. Compositions were prepared using the methods described in Example 1 and according to the formulations (F17-F20) described in Table 6. Where applicable, the particle size and gelation temperature were measured using the methods described in Example 2.

TABLE 6
Composition Containing Other Ingredients. The amount of each
component in each of the formulations is shown (in wt. %).

| | F17 | F18 | F19 | F20 |
|---|---|---|---|---|
| Component | | | | |
| Water | 68.8 | 69.2 | 69.2 | 68.2 |
| Polo407 | 5.88 | 5.75 | 5.75 | 5.67 |
| Polo188 | 0.3 | — | — | — |
| Potassium Sorbate | — | — | — | 0.49 |
| EtOH | 8.4 | 8.33 | 8.33 | 8.21 |
| PEG-40 | 11.1 | 11.1 | 11.11 | 10.95 |
| THC | 5.5 | 5.55 | 5.55 | 5.47 |
| Mucoadhesive | | | | |
| Hypromellose | 0.1 | — | — | — |
| Noveon AA-1 | — | 0.05 | — | — |
| Carbopol 974P | — | — | 0.02 | — |
| Flavorant | | | | |
| Tocobiol | — | — | — | 0.5 |
| Peppermint oils | — | — | — | 0.5 |
| PS | 42 | 42 | — | — |

Polo407 = Poloxamer 407; Polo188 = Poloxamer 188; EtOH = 94% ethanol; PEG-40 = PEG40 hydrogenated castor oil; THC = THC distillate; MCT = MCT oil; GT = gelation temperature (° C.); PS = particle size (nm). A dash indicates that the component was not added, or the characteristic was not measured.

Other examples of implementations will become apparent to the reader in view of the teachings of the present description and as such, will not be further described here.

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As used herein, terms of degree such as “about”, “approximately” and “substantially” mean a reasonable amount of deviation of the modified term such that the end result is not significantly changed. These terms may refer to a measurable value such as an amount, a temporal duration, and the like, and are meant to encompass variations of +/−0.1% of the given value, preferably +/−0.5%, preferably +/−1%, preferably +/−2%, preferably +/−5% or preferably +/−10%.

As used herein, articles such as “a” and “an” when used in a claim, are understood to mean one or more of what is claimed or described.

As used herein, the terms “comprises”, “comprising”, “include”, “includes”, “including”, “contain”, “contains” and “containing” are meant to be non-limiting, i.e., other steps and other sections which do not affect the end of result can be added. The above terms encompass the terms “consisting of” and “consisting essentially of”.

As used herein, the words “preferred”, “preferably” and variants refer to embodiments of the disclosure that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

It is understood that the test methods that are disclosed in the Examples of the present application must be used to determine the respective values of the parameters of Applicant's disclosures as described and claimed herein.

In all embodiments of the present disclosure, all percentages, parts and ratios are based upon the total weight of the compositions of the present disclosure, unless otherwise specified. All such weights as they pertain to listed ingredients are based on the active level and, therefore do not include solvents or by-products that may be included in commercially available materials, unless otherwise specified.

All ratios are weight ratios unless specifically stated otherwise. All temperatures are in Celsius degrees (° C.), unless specifically stated otherwise. All dimensions and values disclosed herein (e.g., quantities, percentages, portions, and proportions) are not to be understood as being strictly limited to the exact numerical values recited. Instead, unless otherwise specified, each such dimension or value is intended to mean both the recited value and a functionally equivalent range surrounding that value. For example, a dimension disclosed as “40 mm” is intended to mean “about 40 mm.”

Note that titles or subtitles may be used throughout the present disclosure for convenience of a reader, but in no way should these limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

Elements of the composition of the disclosure described in connexion with the examples apply mutatis mutandis to other aspects of the disclosure. Therefore, it goes without saying that the compositions of the present disclosure encompass any composition comprising any of the ingredients cited herein, in any embodiment wherein each such ingredient is independently present in any appropriate amount as defined herein. Many such compositions, other than what is specifically set out herein, can be encompassed.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 wt. %” is intended to mean “about 40 wt. %”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the scope of the present disclosure. It is therefore intended to cover in the appended claims all such changes and modifications that are within the scope of this disclosure.

1. A composition comprising:
(i) an emulsion including a cannabinoid component and
(ii) a viscosity modifier, the cannabinoid component including at least one cannabinoid, the composition being in a liquid state at about room temperature, and the viscosity modifier operating to increase a viscosity of the composition such that the composition is in a semi-solid or solid state at about body temperature.

2. A composition according to claim 1, wherein the composition at about body temperature forms a hydrogel.

3. The composition according to claim 1, wherein the viscosity modifier is a mucoadhesive agent operating to increase a viscosity of the composition upon contacting a surface of a mucosa such that the composition forms a hydrogel having mucoadhesive properties.

4. The composition according to claim 1, further comprising
(iii) a pharmaceutically acceptable excipient comprising water.

5. (canceled)

6. The composition according to claim 1, wherein the at least one cannabinoid is an isolated cannabinoid having >75% purity.

7. The composition according to claim 1, wherein the cannabinoid component comprises the at least one cannabinoid in combination with at least one carrier oil.

8. (canceled)

9. The composition according to claim 1, wherein the at least one cannabinoid is cannabidiol (CBD), tetrahydrocannabinol (THC), or a mixture thereof.

10. (canceled)

11. (canceled)

12. (canceled)

13. The composition according to claim 1, wherein the viscosity modifier is a poly(propylene oxide)/poly(ethylene oxide) copolymer.

14. (canceled)

15. The composition according to claim 1, wherein the viscosity modifier comprises a co-polymer selected from the group consisting of: Poloxamer 108, Poloxamer 124, Poloxamer 182, Poloxamer 183, Poloxamer 184, Poloxamer 185, Poloxamer 188, Poloxamer 212, Poloxamer 217, Poloxamer 237, Poloxamer 217, Poloxamer 288, Poloxamer 331, Poloxamer 335, Poloxamer 338, Poloxamer 407 and a mixture thereof.

16. The composition according to claim 15, wherein the viscosity modifier is Poloxamer 407, or a mixture of Poloxamer 407 and Poloxamer 188 where the mixture comprises at least 85 wt. % Poloxamer 407.

17. (canceled)

18. (canceled)

19. The composition according to claim 1, wherein the cannabinoid component and the viscosity modifier are present in a ratio (w/w) of cannabinoid component:viscosity modifier within 0.9-1.1:1-9.5.

20. (canceled)

21. (canceled)

22. (canceled)

23. (canceled)

24. (canceled)

25. (canceled)

26. (canceled)

27. The composition according to claim 1, further comprising at least one surfactant wherein the cannabinoid component and the at least one surfactant are present in a ratio (w/w) of cannabinoid component:surfactant within 0.9-1.1:1.9-2.2.

28. (canceled)

29. (canceled)

30. (canceled)

31. The composition according to claim 1, further comprising at least one permeation enhancer in an amount of from about 0.5 wt. % to about 15 wt. %.

32. (canceled)

33. (canceled)

34. (canceled)

35. (canceled)

36. The composition according to claim 1, further comprising at least one terpene.

37. (canceled)

38. The composition according to claim 1, wherein the composition comprises an oil-in-water emulsion including the cannabinoid component, and wherein the emulsion comprises oil droplets having an average mean particle size of <200 nm.

39. (canceled)

40. The composition according to claim 1, wherein the composition has an initial viscosity of less than about 750 mPas at about room temperature, and an applied viscosity of at least about 5000 mPas at about body temperature.

41. A composition comprising:
a. a cannabinoid component comprising at least one cannabinoid selected from the group consisting of cannabidiol (CBD), tetrahydrocannabinol (THC), and a mixture thereof;
b. a viscosity modifier selected from Poloxamer 407, and a mixture of Poloxamer 407 and Poloxamer 188, where the mixture comprises at least 85% Poloxamer 407, the viscosity modifier present in an amount effective to change the composition from a liquid at about room temperature to a gel upon increase to about body temperature;
c. a surfactant comprising PEG-40 hydrogenated castor oil; and
d. a pharmaceutically acceptable excipient comprising water,
wherein upon application on a mucosa surface, the composition is converted from a liquid to a gel.

42. (canceled)

43. The composition according to claim 41, wherein the cannabinoid component is a mixture of the at least one cannabinoid and MCT oil.

44. (canceled)

45. (canceled)

46. The composition according to claim 41, wherein the cannabinoid component and the viscosity modifier are present in a ratio (w/w) of cannabinoid component:viscosity modifier within 0.9-1.1:1-9.5 wt. %.

47. (canceled)

48. The composition according to claim 41, wherein the cannabinoid component and the surfactant are present in a ratio (w/w) of cannabinoid component:surfactant within 0.9-1.1:1.9-2.2 wt. %.

49. (canceled)

50. (canceled)

51. (canceled)

52. The composition according to claim 41, further comprising at least one permeation enhancer in an amount of from about 0.5 wt. % to about 15 wt. %.

53. (canceled)

54. The composition according to claim 41, wherein the composition comprises an oil-in-water emulsion comprising the cannabinoid component, and wherein the emulsion comprises oil droplets having an average mean particle size of <200 nm.

55. (canceled)

56. A cannabinoid product for transmucosal administration to a subject, wherein the product comprises a composition comprising:
a. an emulsion including a cannabinoid component; and
b. a viscosity modifier, the cannabinoid component including at least one cannabinoid, the composition being in a liquid state at about room temperature, and the viscosity modifier operating to increase a viscosity of the composition such that the composition is in a semi-solid or solid state at about body temperature.

57. The product according to claim 56, wherein the product is adapted for administration of the composition to an oral or nasal mucosa surface of the subject.

58. The product according to claim 56 in one or both of a sublingual liquid spray form and a buccal liquid spray form.

59. (canceled)

60. (canceled)
